SEQUENCE LISTING (2 sequences)

SEQ ID NO: 1 - DNA, 1644 bp, Acinetobacter sp.
atggagattatcatgtcacaaaaaatggattttgatgctatcgtgattggtggtggttttggcggactttatgcagtcaaaaaattaagagacgagctcgaacttaaggttcaggcttttgataaagccacggatgtcgcaggtacttggtactggaaccgttacccaggtgcattgtcggatacagaaacccacctctactgctattcttgggataaagaattactacaatcgctagaaatcaagaaaaaatatgtgcaaggccctgatgtacgcaagtatttacagcaagtggctgaaaagcatgatttaaagaagagctatcaattcaataccgcggttcaatcggctcattacaacgaagcagatgccttgtgggaagtcaccactgaatatggtgataagtacacggcgcgtttcctcatcactgctttaggcttattgtctgcgcctaacttgccaaacatcaaaggcattaatcagtttaaaggtgagctgcatcataccagccgctggccagatgacgtaagttttgaaggtaaacgtgtcggcgtgattggtacgggttccaccggtgttcaggttattacggctgtggcacctctggctaaacacctcactgtcttccagcgttctgcacaatacagcgttccaattggcaatgatccactgtctgaagaagatgttaaaaagatcaaagacaattatgacaaaatttgggatggtgtatggaattcagcccttgcctttggcctgaatgaaagcacagtgccagcaatgagcgtatcagctgaagaacgcaaggcagtttttgaaaaggcatggcaaacaggtggcggtttccgtttcatgtttgaaactttcggtgatattgccaccaatatggaagccaatatcgaagcgcaaaatttcattaagggtaaaattgctgaaatcgtcaaagatccagccattgcacagaagcttatgccacaggatttgtatgcaaaacgtccgttgtgtgacagtggttactacaacacctttaaccgtgacaatgtccgtttagaagatgtgaaagccaatccgattgttgaaattaccgaaaacggtgtgaaactcgaaaatggcgatttcgttgaattagacatgctgatatgtgccacaggttttgatgccgtcgatggcaactatgtgcgcatggacattcaaggtaaaaacggcttggccatgaaagactactggaaagaaggtccgtcgagctatatgggtgtcaccgtaaataactatccaaacatgttcatggtgcttggaccgaatggcccgtttaccaacctgccgccatcaattgaatcacaggtggaatggatcagtgataccattcaatacacggttgaaaacaatgttgaatccattgaagcgacaaaagaagcggaagaacaatggactcaaacttgcgccaatattgcggaaatgaccttattccctaaagcgcaatcctggatttttggtgcgaatatcccgggcaagaaaaacacggtttacttctatctcggtggtttaaaagaatatcgcagtgcgctagccaactgcaaaaaccatgcctatgaaggttttgatattcaattacaacgttcagatatcaagcaacctgccaatgcctaa

SEQ ID NO: 2 - protein, 547 aa, Acinetobacter sp.
MEIIMSQKMDFDAIVIGGGFGGLYAVKKLRDELELKVQAFDKATDVAGTWYWNRYPGALSDTETHLYCYSWDKELLQSLEIKKKYVQGPDVRKYLQQVAEKHDLKKSYQFNTAVQSAHYNEADALWEVTTEYGDKYTARFLITALGLLSAPNLPNIKGINQFKGELHHTSRWPDDVSFEGKRVGVIGTGSTGVQVITAVAPLAKHLTVFQRSAQYSVPIGNDPLSEEDVKKIKDNYDKIWDGVWNSALAFGLNESTVPAMSVSAEERKAVFEKAWQTGGGFRFMFETFGDIATNMEANIEAQNFIKGKIAEIVKDPAIAQKLMPQDLYAKRPLCDSGYYNTFNRDNVRLEDVKANPIVEITENGVKLENGDFVELDMLICATGFDAVDGNYVRMDIQGKNGLAMKDYWKEGPSSYMGVTVNNYPNMFMVLGPNGPFTNLPPSIESQVEWISDTIQYTVENNVESIEATKEAEEQWTQTCANIAEMTLFPKAQSWIFGANIPGKKNTVYFYLGGLKEYRSALANCKNHAYEGFDIQLQRSDIKQPANA

(19) United States
(12) Patent Application Publication
(10) Pub. No.: US 2014/0228595 A1
(43) Pub. Date: Aug. 14, 2014

(54) PROCESSES FOR PRODUCING HEXANEDIOL (HDO), HEXAMETHYLENEDIAMINE (HMD), ADIPONITRILE (ADN), ADIPAMIDE (ADM) AND DERIVATIVES THEREOF

(71) Applicants: Olan S. Fruchey, Hurricane, WV (US); Leo E. Manzer, Wilmington, DE (US); Dilum Dunuwila, Princeton, NJ (US); Brian T. Keen, Pinch, WV (US); Brooke A. Albin, Charleston, WV (US); Nye A. Clinton, Hurricane, WV (US); Bernard D. Dombek, Charleston, WV (US)

(72) Inventors: Olan S. Fruchey, Hurricane, WV (US); Leo E. Manzer, Wilmington, DE (US); Dilum Dunuwila, Princeton, NJ (US); Brian T. Keen, Pinch, WV (US); Brooke A. Albin, Charleston, WV (US); Nye A. Clinton, Hurricane, WV (US); Bernard D. Dombek, Charleston, WV (US)

(73) Assignee: BioAmber S.A.S., Bazancourt (FR)

(21) Appl. No.: 14/125,159

(22) PCT Filed: Sep. 12, 2011
(86) PCT No.: PCT/US2011/051167

§ 371 Date: Feb. 6, 2014

(30) Foreign Application Priority Data
Jun. 10, 2011 (US) PCT.US2011/039923

(51) Int. Cl. C07C 231/02; C07C 209/16

(52) U.S. Cl. CPC C07C 231/02; C07C 209/16
USPC 564/153; 564/478

(57) ABSTRACT

Processes include producing hexanediol (HDO), hexamethylenediamine (HMD), adiponitrile (ADN), adipamide (ADM) and derivatives thereof from fermentation broths containing diammonium adipate (DAA), monoammonium adipate (MAA) and/or adipic acid (AA).

### TECHNICAL FIELD

This disclosure relates to processes for producing nitrogen-containing compounds such as hexanediol (HDO), hexamethylenediamine (HMD), adiponitrile (ADN), adipamide (ADM) and derivatives thereof from fermentation broths containing diammonium adipate (DAA), monoammonium adipate (MAA) and/or adipic acid (AA).

### BACKGROUND

Certain carbonaceous products of sugar fermentation are seen as replacements for petroleum-derived materials for use as feedstocks for the manufacture of carbon-containing chemicals. One such product is MAA. Another such product is AA. Given such a process for the direct production of substantially pure MAA from a DAA, MAA, and/or AA-containing fermentation broth and the possible use of such pure MAA as a source material for the production of HMD, ADN and ADM, it could be helpful to provide processes for producing HMD, ADN and ADM and derivatives thereof in an economic and environmentally friendly way.

### SUMMARY

We provide a process for producing nitrogen-containing compounds including providing a clarified DAA-containing fermentation broth; (a) distilling the broth to form an overhead that includes water and ammonia, and a liquid bottoms that includes MAA, at least some DAA, and at least about 20 wt % water; (b) cooling the bottoms to a temperature sufficient to cause the bottoms to separate into a DAA-containing liquid portion and a MAA-containing solid portion that is substantially free of DAA; (c) separating the solid portion from the liquid portion; (d) (1) contacting at least a part of the solid portion with hydrogen and an ammonia source in the presence of at least one hydrogenation catalyst to produce HMD, (2) dehydrating at least a part of the solid portion in the presence of an ammonia source to produce ADN or (3) dehydrating at least a part of the solid portion in the presence of an ammonia source to produce ADM; and (e) recovering the HMD, ADN or ADM.

We also provide a process for making nitrogen-containing compounds from a DAA-containing fermentation broth, including (a) distilling the broth to form a first overhead that includes water and ammonia, and a first liquid bottoms that includes MAA, at least some DAA, and at least about 20 wt % water; (b) cooling and/or evaporating the bottoms, and optionally adding an antisolvent to the bottoms, to attain a temperature and composition sufficient to cause the bottoms to separate into a DAA-containing liquid portion and a MAA-containing solid portion that is substantially free of DAA; (c) separating the solid portion from the liquid portion; (d) recovering the solid portion; (e) dissolving the solid portion in water to produce an aqueous MAA solution; (f) distilling the aqueous MAA solution at a temperature and pressure sufficient to form a second overhead that includes water and ammonia, and a second bottoms that includes a major portion of AA, a minor portion of MAA, and water; (g) cooling and/or evaporating the second bottoms to cause the second bottoms to separate into a second liquid portion in contact with a second solid portion that preferably consists essentially of AA and is substantially free of MAA; (h) separating the second solid portion from the second liquid portion; (i) recovering the second solid portion; (j) (1) contacting at least a part of the second solid portion with hydrogen and an ammonia source in the presence of at least one hydrogenation catalyst to produce HMD, (2) dehydrating at least a part of the second solid portion in the presence of an ammonia source to produce ADN or (3) dehydrating at least a part of the second solid portion in the presence of an ammonia source to produce ADM; and (k) recovering the HMD, AND or ADM.

We further provide a process for making nitrogen-containing compounds from a clarified MAA-containing broth including (a) optionally, adding MAA, DAA, AA, NH₃, and/or NH₄⁺ to the broth to preferably maintain the pH of the broth below 6; (b) distilling the broth to form an overhead that includes water and optionally ammonia, and a liquid bottoms that includes MAA, at least some DAA, and at least about 20 wt % water; (c) cooling and/or evaporating the bottoms, and optionally adding an antisolvent to the bottoms, to attain a temperature and composition sufficient to cause the bottoms to separate into a DAA-containing liquid portion and a MAA-containing solid portion that is substantially free of DAA; (d) separating the solid portion from the liquid portion; (e) recovering the solid portion; (f) (1) contacting at least a part of the second solid portion with hydrogen and an ammonia source in the presence of at least one hydrogenation catalyst to produce HMD, (2) dehydrating at least a part of the second solid portion in the presence of an ammonia source to produce ADN or (3) dehydrating at least a part of the second solid portion in the presence of an ammonia source to produce ADM; and (g) recovering the HMD, AND or ADM.

We further yet provide a process for making nitrogen-containing compounds from a clarified MAA-containing fermentation broth including (a) optionally, adding MAA, DAA, AA, NH₃, and/or NH₄⁺ to the broth to preferably maintain the pH of the broth below 6; (b) distilling the broth to form an overhead that includes water and optionally ammonia, and a liquid bottoms that includes MAA, at least some DAA, and at least about 20 wt % water; (c) cooling and/or evaporating the bottoms, and optionally adding an antisolvent to the bottoms, to attain a temperature and composition sufficient to cause the bottoms to separate into a DAA-containing liquid portion and a MAA-containing solid portion that is substantially free of DAA; (d) separating the solid portion from the liquid portion; and (e) recovering the solid portion; (f) dissolving the solid portion in water to produce an aqueous MAA solution; (g) distilling the aqueous MAA solution at a temperature and pressure sufficient to form a second overhead that includes water and ammonia, and a second bottoms that includes a major portion of AA, a minor portion of MAA, and water; (h) cooling and/or evaporating the second bottoms to cause the second bottoms to separate into a second liquid portion in contact with a second solid portion that preferably consists essentially of AA and is substantially free of MAA; (i) separating the second solid portion from the second liquid portion; (j) recovering the second solid portion; (k) (1) contacting at least a part of the second solid portion with hydrogen and an ammonia source in the presence of at least one hydrogenation catalyst to produce HMD, (2) dehydrating at least a part of the second solid portion in the presence of an ammonia source to produce ADN or (3) dehydrating at least a part of the second solid portion in the presence of an ammonia source to produce ADM; and (l) recovering the HMD, AND or ADM.

We further provide a process for making nitrogen containing compounds including indirect conversion of AA to HMD by hydrogenation of AA to hexanediol (HDO) and amination of HDO to HMD.

### BRIEF DESCRIPTION OF THE DRAWINGS

FIG. 1 is a block diagram of a bioprocessing system.

FIG. 2 is a graph showing the solubility of MAA as a function of temperature in both water and a 30% aqueous DAA solution.

FIG. 3 is a flow diagram showing the selected production of HMD, ADN, ADM and derivatives thereof from MAA.

FIG. 4 is a flow diagram showing the selected production of HDO, HMD, ADN, ADM and derivatives thereof from AA.

FIG. 5 is a flow diagram showing the selected production of HMD, ADN, ADM and derivatives thereof from DAA.

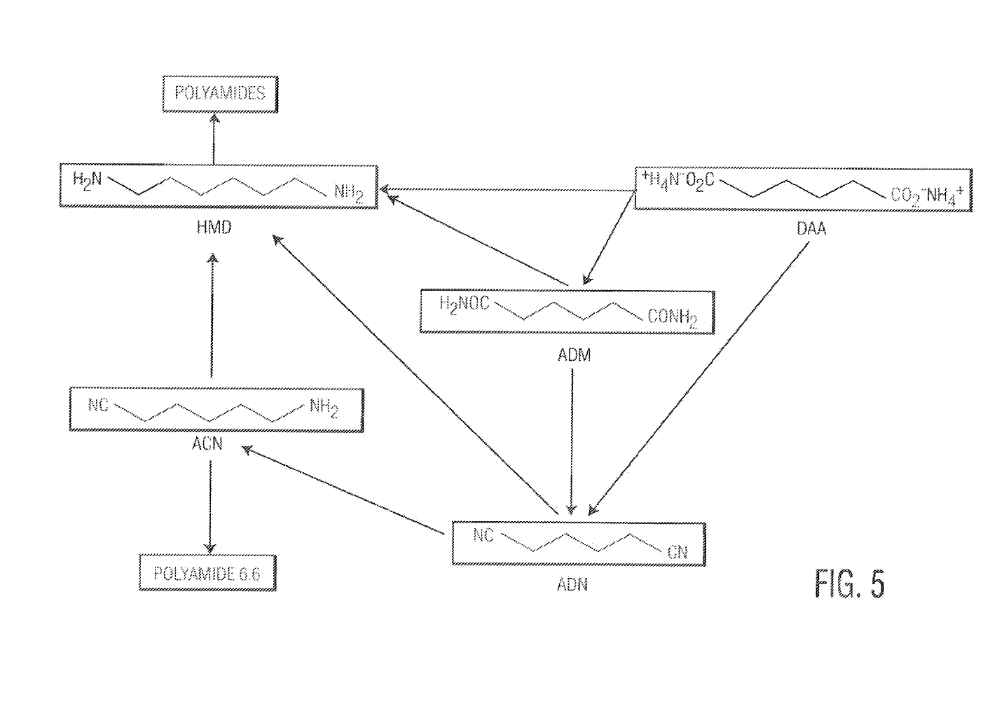

### DETAILED DESCRIPTION

It will be appreciated that at least a portion of the following description is intended to refer to representative examples of processes selected for illustration in the drawings and is not intended to define or limit the disclosure, other than in the appended claims.

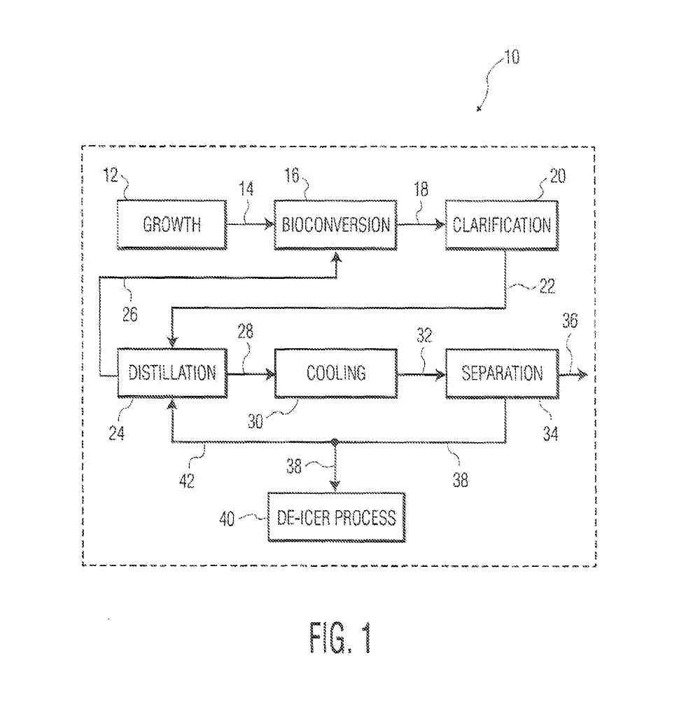

Our processes may be appreciated by reference to FIG. 1, which shows in block diagram form one representative example 10 of our methods.

A growth vessel 12, typically an in-place steam sterilizable fermentor, may be used to grow a microbial culture (not shown) that is subsequently utilized for the production of the DAA, MAA, and/or AA-containing fermentation broth. Such growth vessels are known in the art and are not further discussed.

The microbial culture may comprise microorganisms capable of producing AA from fermentable carbon sources such as carbohydrate sugars (e.g., glucose), cyclohexanol, alkanes (e.g., n-alkanes) and plant based oils. Representative examples of microorganisms include Escherichia coli (E. coli), Aspergillus niger, Corynebacterium glutamicum (also called Brevibacterium flavum), Enterococcus faecalis, Veillonella parvula, Actinobacillus succinogenes, Paecilomyces varioli, Saccharomyces cerevisiae, Candida tropicalis, Bacteroides fragilis, Bacteroides ruminicola, Bacteroides amylophilus, Klebsiella pneumoniae, mixtures thereof and the like.

Preferred microorganisms for include the Candida tropicalis (Castellani) Berkhout, anamorph strain OH23 having ATCC accession number 24887, E. coli strain AB2834/pKD136/pKD8.243A/pKD8.292 having ATCC accession number 69875, the E. coli cosmid clones designated 5B12, 5F5, 8F6 and 14D7 comprising a vector expressing the cyclohexanone monoxygenase having the amino acid sequence shown in SEQ ID NO: 2 and encoded by SEQ ID NO: 1 from Acinetobacter strain SE19, and the yeast strain available from Verdezyne, Inc. (Carslbad, Calif., USA; hereinafter “Verdezyne Yeast”) which produces AA from alkanes and other carbon sources.

Fermentation broths containing AA can be produced from the Candida tropicalis (Castellani) Berkhout, anamorph strain OH23 having ATCC accession number 24887 by culture at 32 C in a liquid medium containing 300 mg of NH₄H₂PO₄, 200 mg of KH₂PO₄, 100 mg of K₂HPO₄, 50 mg of MgSO₄·7H₂O, 1 μg of biotin, 0.1% (w/v) yeast extract and about 1% (v/v) n-hexadecane in 100 ml of distilled water. Other culture media such as YM broth containing n-hexadecane may also be used. The procedure for producing fermentation broths containing AA from media containing n-hexadecane by culturing Candida tropicalis (Castellani) Berkhout, anamorph strain OH23 having ATCC accession number 24887 is also described in Okuhura et al., 35 Agr. Biol. Chem. 1376 (1971) the subject matter of which is incorporated herein by reference.

Fermentation broths containing AA can also be produced from E. coli strain AB2834/pKD136/pKD8.243A/pKD8.292 having ATCC accession number 69875. This can be done as follows. One liter of LB medium (in 4 L Erlenmeyer shake flask) containing IPTG (0.2 mM), ampicillin (0.05 g), chloramphenicol (0.02 g) and spectinomycin (0.05 g) can be inoculated with 10 mL of an overnight culture of E. coli strain AB2834/pKD136/pKD8.243A/pKD8.292 cells grown at 250 rpm for 10 h at 37 C. The cells can be harvested, resuspended in 1 L of M9 minimal medium containing 56 mM D-glucose, shikimic acid (0.04 g), IPTG (0.2 mM), ampicillin (0.05 g), chloramphenicol (0.02 g) and spectinomycin (0.05 g). The cultures can then be returned to 37 C incubation. After resuspension in minimal medium the pH of the culture can be closely monitored, particularly over the initial 12 h. When the culture reaches a pH of 6.5, 5N NaOH or an appropriate amount of another base such as ammonium hydroxide can be added to adjust the pH back to approximately 6.8. Over the 48 h accumulation period, the culture should not allowed to fall below pH 6.3. After 24 h in the medium 12 mM cis, cis-muconate and 1 mM protocatechuate may be detected in the culture supernatant along with 23 mM D-glucose. After 48 h in the medium E. coli strain AB2834/pKD136/pKD38.243A/pKD8.292 cells can essentially replace the 56 mM D-glucose in the medium with 17 mM cis, cis-muconate.

The reduction of microbially synthesized cis, cis-muconate AA to produce a fermentation broth containing AA can then proceed as follows. Fifty milligrams of platinum on carbon (10%) can be added to 6 mL of a cell-free culture supernatant from the fermentation containing about 17.2 mM cis, cis-muconate. This sample can then be hydrogenated at 50 psi hydrogen pressure for 3 h at room temperature to produce a fermentation broth containing AA. The fermentation broth produced in this fashion may contain, for example, about 15.1 mM AA. The procedure for producing fermentation broths containing AA by culturing E. coli strain AB2834/pKD136/pKD8.243A/pKD8.292 cells by culture in a growth medium comprising D-glucose is also described in Draths & Frost, 116 J. Am. Chem. Soc. 399 (1994); Draths and Frost, 18 Biotechnol. Prog. 201 (2002); U.S. Pat. No. 5,487,987 and U.S. Pat. No. 5,616,496 the subject matter of which is incorporated herein by reference.

Fermentation broths containing AA can also be produced from the E. coli cosmid clones designated 5B12, 5F5, 8F6 and 14D7 comprising a vector expressing the cyclohexanone monoxygenase SEQ ID NO: 2 encoded by SEQ ID NO: 1 from Acinetobacter strain SE19 by culturing these clones in M9 minimal medium supplemented with 0.4% glucose as the carbon source. Cells can be grown at 30 C with shaking for 2 h and the addition of 330 ppm of cyclohexanol to the medium. This can be followed by further incubation at 30 C for an additional period of time such as, for example, 2 h, 4 h or 20 h or other time intervals. The procedure for producing fermentation broths containing AA by culturing the E. coli cosmid clones designated 5B12, 5F5, 8F6 and 14D7 comprising a vector expressing the cyclohexanone monoxygenase encoded by SEQ ID NO: 1 from Acinetobacter strain SE19 in a growth medium comprising D-glucose and clyclohexanol is also described in U.S. Pat. No. 6,794,165 the subject matter of which is incorporated herein by reference.

Fermentation broths containing AA can also be produced with the Verdezyne Yeast strain available from Verdezyne, Inc. (Carsibad, Calif., USA) which was reported on Feb. 8, 2010 to produce AA when cultured in a medium (e.g., SD medium) comprising alkanes or other carbon sources such as sugars and plant-based oils.

Fermentation broths containing AA can also be produced from E. coli or other microorganisms transformed with nucleic acids encoding succinyl-CoA:acetyl-CoA acyl transferase; 3-hydroxyacyl-CoA dehydrogenase; 3-hydroxyadipyl-CoA dehydratase; 5-carboxy-2-pentenoyl-CoA reductase; adipyl-CoA synthetase, phosphotransadipylase/adipate kinase, adipyl-CoA transferase or adipyl-CoA hydrolase. Fermentation broths containing AA can also be produced from E. coli or other microorganisms transformed with nucleic acids encoding succinyl-CoA:acetyl-CoA acyl transferase; 3-oxoadipyl-CoA transferase; 3-oxoadipate reductase; 3-hydroxyadipate dehydratase; and 2-enoate reductase. Fermentation broths containing AA can also be produced from E. coli or other microorganisms transformed with nucleic acids encoding alpha-ketoadipyl-CoA synthetase, phosphotransketoadipylase/alpha-ketoadipate kinase or alpha-ketoadipyl-CoA:acetyl-CoA tranferase; 2-hydroxyadipyl-CoA dehydrogenase; 2-hydroxyadipyl-CoA dehydratase; 5-carboxy-2-penteoyl-CoA reductase; and adipyl-CoA synthetase, phosphotransadipylase/adipate kinase, adipyl-CoA:acetyl-CoA transferase or adipyl-CoA hydrolase. Fermentation broths containing AA can also be produced from E. coli or other microorganisms transformed with nucleic acids encoding 2-hydroxyadipate dehydrogenase; 2-hydroxyadipyl-CoA synthetase, phosphotranshydroxyadipylase/2-hydroxyadipate kinase or 2-hydroxyadipyl-CoA:acetyl-CoA transferase; 2-hydroxyadipyl-CoA dehydratase; 5-carboxy-2-pentenoyl-CoA reductase; and adipyl-CoA synthetase, phosphotransadipylase/adipate kinase, adipyl-CoA:acetyl-CoA transferase or adipyl-CoA hydrolase. Fermentations with E. coli or other microorganisms transformed with nucleic acids encoding these enzymes may be performed using a variety of different carbon sources under standard conditions in standard culture mediums (e.g., M9 minimal medium) and appropriate antibiotic or nutritional supplements necessary to maintain the transformed phenotype. The procedure for producing fermentation broths containing AA by culturing E. coli or other microorganisms transformed with nucleic acids encoding these enzymes, appropriate growth mediums and carbon sources are also described in US 2009/0305364, the subject matter of which is incorporated herein by reference.

Procedures for producing fermentation broths containing dicarboxylic acids such as AA by culturing Saccharomyces cerevisiae strains, and other microorganism strains, appropriate growth mediums and carbon sources are also described in WO 2010/003728, the subject matter of which is incorporated herein by reference.

A fermentable carbon source (e.g., carbohydrates and sugars), optionally a source of nitrogen and complex nutrients (e.g., corn steep liquor), additional media components such as vitamins, salts and other materials that can improve cellular growth and/or product formation, and water may be fed to the growth vessel 12 for growth and sustenance of the microbial culture. Typically, the microbial culture is grown under aerobic conditions provided by sparging an oxygen-rich gas (e.g., air or the like). Typically, an acid (e.g., sulphuric acid or the like) and ammonium hydroxide are provided for pH control during the growth of the microbial culture.

In one example (not shown), the aerobic conditions in growth vessel 12 (provided by sparging an oxygen-rich gas) are switched to anaerobic conditions by changing the oxygen-rich gas to an oxygen-deficient gas (e.g., CO₂ or the like). The anaerobic environment may trigger bioconversion of the fermentable carbon source to AA in situ in growth vessel 12. Ammonium hydroxide is provided for pH control during bioconversion of the fermentable carbon source to AA. The produced AA is at least partially neutralized to DAA due to the presence of the ammonium hydroxide, leading to the production of a broth comprising DAA. The CO₂ provides an additional source of carbon for the production of AA.

In another example, the contents of growth vessel 12 may be transferred via stream 14 to a separate bioconversion vessel 16 for bioconversion of a carbohydrate source to AA. An oxygen-deficient gas (e.g., CO₂ or the like) may be sparged in bioconversion vessel 16 to provide anaerobic conditions that trigger production of AA. Ammonium hydroxide is provided for pH control during bioconversion of the carbohydrate source to AA. Due to the presence of the ammonium hydroxide, the AA produced is at least partially neutralized to DAA, leading to production of a broth that comprises DAA. The CO₂ provides an additional source of carbon for production of AA.

In another example, the bioconversion may be conducted at relatively low pH (e.g., 3-6). A base (ammonium hydroxide or ammonia) may be provided for pH control during bioconversion of the carbohydrate source to AA. Depending of the desired pH, due to the presence or lack of the ammonium hydroxide, either AA is produced or the AA produced is at least partially neutralized to MAA, DAA, or a mixture comprising AA, MAA and/or DAA. Thus, the AA produced during bioconversion can be subsequently neutralized, optionally in an additional step, by providing either ammonia or ammonium hydroxide leading to a broth comprising DAA. As a consequence, a “DAA-containing fermentation broth” generally means that the fermentation broth comprises DAA and possibly any number of other components such as MAA and/or AA, whether added and/or produced by bioconversion or otherwise. Similarly, a “MAA-containing fermentation broth” generally means that the fermentation broth comprises MAA and possibly any number of other components such as DAA and/or AA, whether added and/or produced by bioconversion or otherwise.

The broth resulting from the bioconversion of the fermentable carbon source (in either growth vessel 12 or bioconversion vessel 16, depending on where the bioconversion takes place), typically contains insoluble solids such as cellular biomass and other suspended material, which are transferred via stream 18 to clarification apparatus 20 before distillation. Removal of insoluble solids clarifies the broth. This reduces or prevents fouling of subsequent distillation equipment. The insoluble solids can be removed by any one of several solid-liquid separation techniques, alone or in combination, including but not limited to centrifugation and filtration (including, but not limited to ultra-filtration, micro-filtration or depth filtration). The choice of filtration technique can be made using techniques known in the art. Soluble inorganic compounds can be removed by any number of known methods such as, but not limited to, ion exchange and physical adsorption.

An example of centrifugation is a continuous disc stack centrifuge. It may be useful to add a polishing filtration step following centrifugation such as dead-end or cross-flow filtration, which may include the use of a filter aide such as diatomaceous earth or the like, or more preferably ultra-filtration or micro-filtration. The ultra-filtration or micro-filtration membrane can be ceramic or polymeric, for example. One example of a polymeric membrane is SelRO MPS-U20P (pH stable ultra-filtration membrane) manufactured by Koch Membrane Systems (850 Main Street, Wilmington, Mass., USA). This is a commercially available polyethersulfone membrane with a 25,000 Dalton molecular weight cut-off which typically operates at pressures of 0.35 to 1.38 MPa (maximum pressure of 1.55 MPa) and at temperatures up to 50° C. Alternatively, a filtration step may be employed, such as ultra-filtration or micro-filtration alone.

The resulting clarified DAA-containing broth, substantially free of the microbial culture and other solids, is transferred via stream 22 to distillation apparatus 24.

The clarified distillation broth should contain DAA and/or MAA in an amount that is at least a majority of, preferably at least about 70 wt %, more preferably 80 wt % and most preferably at least about 90 wt % of all the diammonium dicarboxylate salts in the broth. The concentration of DAA and/or MAA as a weight percent (wt %) of the total dicarboxylic acid salts in the fermentation broth can be easily determined by high pressure liquid chromatography (HPLC) or other known means.

Water and ammonia are removed from distillation apparatus 24 as an overhead, and at least a portion is optionally recycled via stream 26 to bioconversion vessel 16 (or growth vessel 12 operated in the anaerobic mode). Distillation temperature and pressure are not critical as long as the distillation is carried out in a way that ensures that the distillation overhead contains water and ammonia, and the distillation bottoms comprises at least some DAA and at least about 20 wt % water. A more preferred amount of water is at least about 30 wt % and an even more preferred amount is at least about 40 wt %. The rate of ammonia removal from the distillation step increases with increasing temperature and also can be increased by injecting steam (not shown) during distillation. The rate of ammonia removal during distillation may also be increased by conducting distillation under a vacuum or by sparging the distillation apparatus with a non-reactive gas such as air, nitrogen or the like.

Removal of water during the distillation step can be enhanced by the use of an organic azeotroping agent such as toluene, xylene, hexane, cyclohexane, methyl cyclohexane, methyl isobutyl ketone, heptane or the like, provided that the bottoms contains at least about 20 wt % water. If the distillation is carried out in the presence of an organic agent capable of forming an azeotrope consisting of the water and the agent, distillation produces a biphasic bottoms that comprises an aqueous phase and an organic phase, in which case the aqueous phase can be separated from the organic phase, and the aqueous phase used as the distillation bottoms. By-products such as adipamide and adipimide are substantially avoided provided the water level in the bottoms is maintained at a level of at least about 30 wt %.

A preferred temperature for the distillation step is in the range of about 50 to about 300° C., depending on the pressure. A more preferred temperature range is about 90 to about 150° C., depending on the pressure. A distillation temperature of about 110 to about 140° C. is preferred. “Distillation temperature” refers to the temperature of the bottoms (for batch distillations this may be the temperature at the time when the last desired amount of overhead is taken).

Adding a water miscible organic solvent or an ammonia separating solvent facilitates deammoniation over a variety of distillation temperatures and pressures as discussed above. Such solvents include aprotic, bipolar, oxygen-containing solvents that may be able to form passive hydrogen bonds. Examples include, but are not limited to, diglyme, triglyme, tetraglyme, sulfoxides such as dimethylsulfoxide (DMSO), amides such as dimethylformamide (DMF) and dimethylacetamide, sulfones such as dimethylsulfone, sulfolane, polyethyleneglycol (PEG), butoxytriglycol, N-methylpyrolidone (NMP), ethers such as dioxane, methyl ethyl ketone (MEK) and the like. Such solvents aid in the removal of ammonia from the DAA in the clarified broth. Regardless of the distillation technique, it is important that the distillation be carried out in a way that ensures that at least some DAA and at least about 20 wt % water remain in the bottoms and even more advantageously at least about 30 wt %.

The distillation can be performed at atmospheric, sub-atmospheric or super-atmospheric pressures. The distillation can be a one-stage flash, a multistage distillation (i.e., a multistage column distillation) or the like. The one-stage flash can be conducted in any type of flasher (e.g., a wiped film evaporator, thin film evaporator, thermosiphon flasher, forced circulation flasher and the like). The multistages of the distillation column can be achieved by using trays, packing or the like. The packing can be random packing (e.g., Raschig rings, Pall rings, Berl saddles and the like) or structured packing (e.g., Koch-Sulzer packing, Intalox packing, Mellapak and the like). The trays can be of any design (e.g., sieve trays, valve trays, bubble-cap trays and the like). The distillation can be performed with any number of theoretical stages.

If the distillation apparatus is a column, the configuration is not particularly critical, and the column can be designed using well known criteria. The column can be operated in either stripping mode, rectifying mode or fractionation mode. Distillation can be conducted in either batch or continuous mode. In the continuous mode, the broth is fed continuously into the distillation apparatus, and the overhead and bottoms are continuously removed from the apparatus as they are formed. The distillate from distillation is an ammonia/water solution, and the distillation bottoms is a liquid, aqueous solution of MAA and DAA, which may also contain other fermentation by-product salts (i.e., ammonium acetate, ammonium formate, ammonium lactate and the like) and color bodies.

The distillation bottoms can be transferred via stream 28 to cooling apparatus 30 and cooled by conventional techniques. Cooling technique is not critical. A heat exchanger (with heat recovery) can be used. A flash vaporization cooler can be used to cool the bottoms down to about 15° C. Cooling to below 15° C. typically employs a refrigerated coolant such as, for example, glycol solution or, less preferably, brine. A concentration step can be included prior to cooling to help increase product yield. Further, both concentration and cooling can be combined using methods known such as vacuum evaporation and heat removal using integrated cooling jackets and/or external heat exchangers.

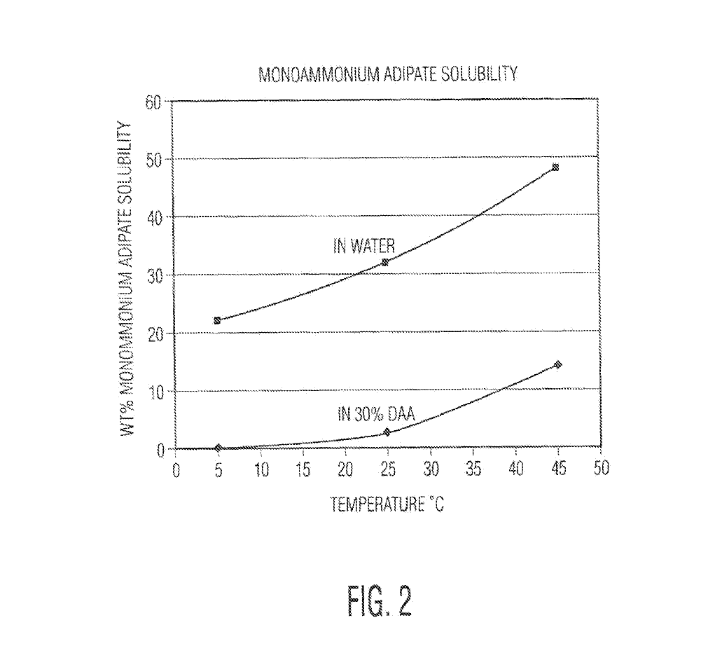

We found that the presence of some DAA in the liquid bottoms facilitates cooling-induced separation of the bottoms into a liquid portion in contact with a solid portion that at least “consists essentially” of MAA (meaning that the solid portion is at least substantially pure crystalline MAA) by reducing the solubility of MAA in the liquid, aqueous, DAA-containing bottoms. FIG. 2 illustrates the reduced solubility of MAA in an aqueous 30 wt % DAA solution at various temperatures ranging from 0 to 60° C. The upper curve shows that even at 0° C. MAA remains significantly soluble in water (i.e., about 20 wt % in aqueous solution). The lower curve shows that at 0° C. MAA is essentially insoluble in a 30 wt % aqueous DAA solution. We discovered, therefore, that MAA can be more completely crystallized out of an aqueous solution if some DAA is also present in that solution. A preferred concentration of DAA in such a solution is about 30 wt %. A more preferred concentration of DAA in such a solution is in the ppm to about 3 wt % range. This phenomenon allows crystallization of MAA (i.e., formation of the solid portion of the distillation bottoms) at temperatures higher than those that would be required in the absence of DAA.

When about 50% of the ammonia is removed from DAA contained in an aqueous medium the adipate species establish an equilibrium molar distribution that is about 0.2:0.6:0.2 in DAA:MAA:AA within a pH range of 4.9 to 5.1, depending on the operating temperature and pressure. When this composition is concentrated and cooled, MAA exceeds its solubility limit in water and crystallizes. When MAA undergoes a phase change to the solid phase, the liquid phase equilibrium resets thereby producing more MAA (DAA donates an ammonium ion to AA). This causes more MAA to crystallize from solution and continues until appreciable quantities of AA are exhausted and the pH tends to rise. As the pH rises, the liquid phase distribution favors DAA. However, since DAA is highly soluble in water, MAA continues to crystallize as its solubility is lower than DAA. In effect, the liquid phase equilibrium and the liquid-solid equilibria of adipate species act as a “pump” for MAA crystallization, thereby enabling MAA crystallization in high yield.

In addition to cooling, evaporation, or evaporative cooling described above, crystallization of MAA can be enabled and/or facilitated by addition of an antisolvent. In this context, antisolvents may be solvents typically miscible with water, but cause crystallization of a water soluble salt such as MAA due to lower solubility of the salt in the solvent. Solvents with an antisolvent effect on MAA can be alcohols such as ethanol and propanol, ketones such as methyl ethyl ketone, ethers such as tetrahydrofuran and the like. The use of antisolvents is known and can be used in combination with cooling and evaporation or separately.

The distillation bottoms, after cooling in unit 30, is fed via stream 32 to separator 34 for separation of the solid portion from the liquid portion. Separation can be accomplished via pressure filtration (e.g., using Nutsche or Rosenmond type pressure filters), centrifugation and the like. The resulting solid product can be recovered as product 36 and dried, if desired, by standard methods.

After separation, it may be desirable to treat the solid portion to ensure that no liquid portion remains on the surface(s) of the solid portion. One way to minimize the amount of liquid portion that remains on the surface of the solid portion is to wash the separated solid portion with water and dry the resulting washed solid portion (not shown). A convenient way to wash the solid portion is to use a so-called “basket centrifuge” (not shown). Suitable basket centrifuges are available from The Western States Machine Company (Hamilton, Ohio, USA).

The liquid portion of the separator 34 (i.e., the mother liquor) may contain remaining dissolved MAA, any unconverted DAA, any fermentation by-products such as ammonium acetate, lactate, or formate, and other minor impurities. This liquid portion can be fed via stream 38 to a downstream apparatus 40. In one instance, apparatus 40 may be a means for making a de-icer by treating the mixture with an appropriate amount of potassium hydroxide, for example, to convert the ammonium salts to potassium salts. Ammonia generated in this reaction can be recovered for reuse in the bioconversion vessel 16 (or growth vessel 12 operating in the anaerobic mode). The resulting mixture of potassium salts is valuable as a de-icer and anti-icer.

The mother liquor from the solids separation step 34, can be recycled (or partially recycled) to distillation apparatus 24 via stream 42 to further enhance recovery of MAA, as well as further convert DAA to MAA.

The solid portion of the cooling-induced crystallization is substantially pure MAA and is, therefore, useful for the known utilities of MAA. One such use is for the production of products such as HMD, ADN, ADM and derivatives thereof.

HPLC can be used to detect the presence of nitrogen-containing impurities such as adipamide and adipimide. The purity of MAA can be determined by elemental carbon and nitrogen analysis. An ammonia electrode can be used to determine a crude approximation of MAA purity.

Depending on the circumstances and various operating inputs, there are instances when the fermentation broth may be a clarified MAA-containing fermentation broth or a clarified AA-containing fermentation broth. In those circumstances, it can be advantageous to add MAA, DAA and/or AA to those fermentation broths to facilitate the production of substantially pure MAA. For example, the operating pH of the fermentation broth may be oriented such that the broth is a MAA-containing broth or a AA-containing broth. MAA, DAA, AA, ammonia and/or ammonium hydroxide may optionally be added to those broths to attain a broth pH preferably less than 6, optionally along with changing the ammonium balance to facilitate production of the above-mentioned substantially pure MAA. Also, it is possible that MAA, DAA and/or AA from other sources may be added as desired. In one particular form, it is especially advantageous to recycle MAA, DAA and water from the liquid bottoms resulting from the distillation step 24 and/or the liquid portion from the separator 34 into the fermentation broth. In referring to the MAA-containing broth, such broth generally means that the fermentation broth comprises MAA and possibly any number of other components such as DAA and/or AA, whether added and/or produced by bioconversion or otherwise.

The solid portion can be converted into AA by removing ammonia. This can be carried out as follows. The solid portion (consisting essentially of MAA) obtained from any of the above-described conversion processes can be dissolved in water to produce an aqueous MAA solution. This solution can then be distilled at a temperature and pressure sufficient to form an overhead that comprises water and ammonia, and a bottoms that comprises a major portion of AA, a minor portion of MAA and water. The bottoms can be cooled to cause it to separate into a liquid portion in contact with a solid portion that consists essentially of AA and is substantially free of MAA. The solid portion can be separated from the second liquid portion and recovered as substantially pure AA as determined by HPLC.

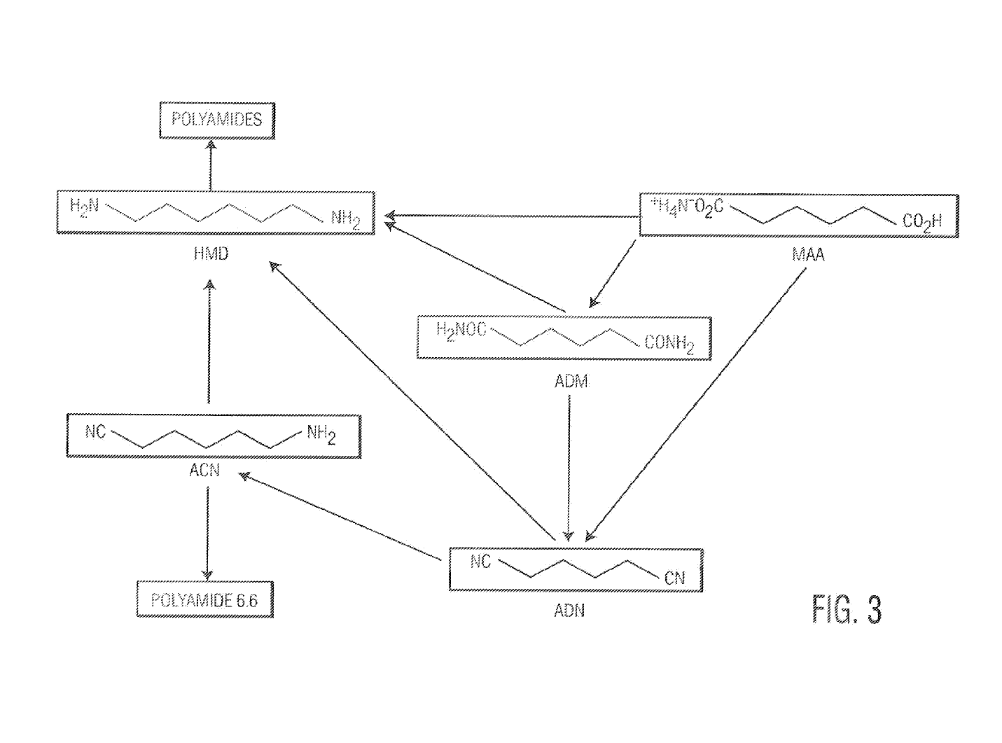

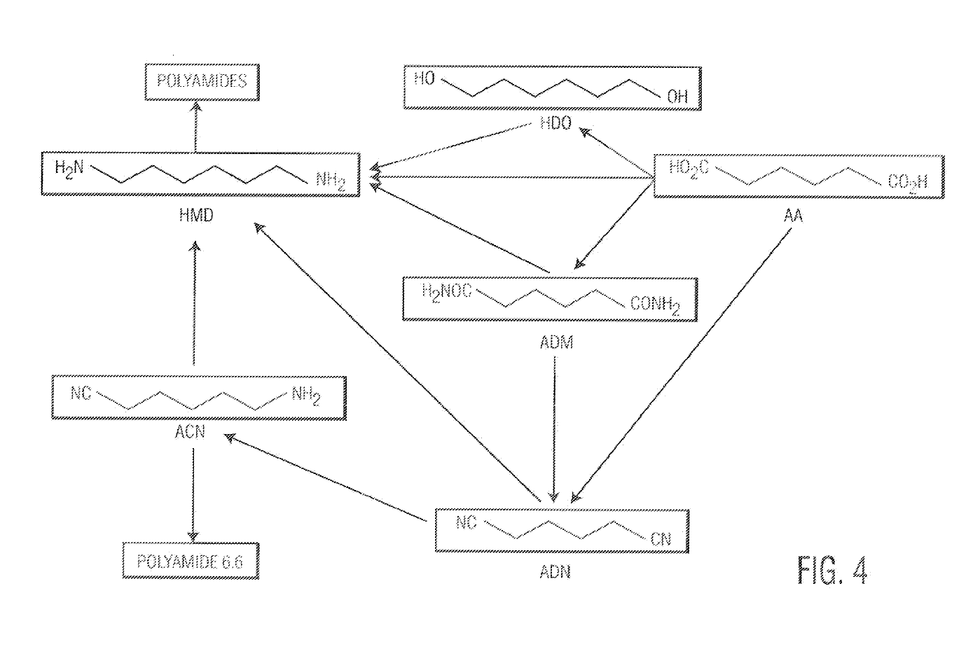

Streams comprising AA, MAA and/or DAA as described above may be converted to selected downstream products such as HMD, ADN, 6-aminocapronitrile (ACN), ADM and the like as described below as shown in FIG. 3 and FIG. 4. In initiating those processes, typically the AA, MAA and/or DAA may be dissolved in water to form an aqueous solution thereof which can be directly fed to the downstream reactor.

The AA, MAA or DAA may be converted to ADN, either directly or indirectly through the intermediate ADM by dehydration. Such dehydrations may be achieved thermally, enzymatically or in the presence of catalysts. Thus, appropriate temperatures, pressures and catalysts are selected to achieve the appropriate level of dehydration, depending on whether the conversion to ADN occurs directly or indirectly.

For example, the conversion can employ an appropriate dehydrating catalyst such as acidic or basic catalysts, including phosphates as disclosed in U.S. Pat. No. 4,237,067 and supported catalysts utilizing Ti, V, Hf or Zr on clays or alumina as disclosed in U.S. Pat. No. 5,587,498. Such catalysts are typically employed at temperatures of about 220° C. to about 350° C. at pressures of about 1.172 to 4.37 MPa, for example.

Alternatively, dehydration can be achieved thermally as disclosed in U.S. Pat. No. 3,296,303, wherein acids plus an ammonia source are thermally dehydrated in the presence of glycol solvents at temperatures of about 100° C. to 130° C. at pressures of about 1.03 to about 1.38 MPa.

As a consequence, AA, MAA or DAA may be dehydrated directly to ADN or indirectly to ADN by the intermediate ADM. Then, once ADN is produced, it is possible to convert ADN directly to an amine such as HMD or to indirectly convert ADN to HMD through the intermediate ACN.

For example, direct conversion from ADN to HMD can be achieved in any number of ways such as disclosed in U.S. Pat. No. 6,376,714 wherein dinitriles in the presence of hydrogen and an ammonia source are converted utilizing catalysts such as Fe, Co, Ni, Rh or Pd promoted with Ru, Cr or W at temperatures of about 50° C. to about 150° C. at about 2.01 to about 10.34 MPa. The result is high yields of the diamine, in this case HMD.

Similarly, U.S. Pat. No. 4,003,933 converts nitriles to amines with hydrogen over a Co/ZrO₂ catalyst at about 120° C. to about 130° C. and at about 10.34 MPa. Other catalysts may include Fe, Rh, Ir and Pt on TiO₂ or ZrO₂.

The indirect conversion of ADN to ACN can be achieved by selecting appropriate hydrogenation conditions such as those disclosed in U.S. Pat. No. 5,151,543 wherein nitriles are converted to amino nitriles, in this case ADN to ACN, utilizing Raney catalysts such as Co or Ni promoted with Fe, Cr or Mo with hydrogen and an ammonia source at about 50° C. to about 80° C. at pressures of 1.72 to 6.89 MPa.

Similarly, the amino nitrile or diamino compounds can be co-produced from the dinitriles such as those disclosed in U.S. Pat. No. 7,132,562. US '562 utilizes Fe, Co, Ru, Ni catalysts modified with Cr, V, Ti or Mn at temperatures of about 50° C. to about 250° C. and 20.68 to 34.47 MPa to achieve high yields and selectivity to the diamine or amino nitrile. The catalysts may also be modified with ordinary P or N with HCN, or CO and hydrogen and an ammonia source.

It is also possible to convert AA, MAA or DAA directly to diamines such as HMD either directly or indirectly through ADM. For example, U.S. Pat. No. 2,223,303 discloses the conversion of AA and ADM to HMD with hydrogen and an ammonia source or alkyl amines with a Cd or Cu catalyst at temperatures of about 200° C. to about 450° C. at pressures of about 1.01 to 30.4 MPa. Similarly, U.S. Pat. No. 3,579,583 discloses the conversion of dicarboxylic acids to amines, particularly alkyl amines, utilizing hydrogen and an ammonia source at temperatures of 200° C. to 300° C. at pressures of 10.1 to 30.4 MPa in the presence of a Zn—Al₂O₃ or Zn—Cr catalyst.

Further, U.S. Pat. No. 4,935,546 discloses the conversion of acids to amines with hydrogen and an ammonia source in the presence of a Co, Cu or Cr catalyst on a TiO₂ or Al₂O₃ support at temperatures of 250° C. to 350° C. and at pressures of 2 to 15 MPa.

Indirect conversion of AA to HMD may also be achieved by hydrogenation of AA to HDO, as shown in FIG. 4. Amination of HDO to HMD can be conducted by the use of a suitable catalyst and an ammonia source. For example, suitable catalysts and reaction conditions for amination of HDO include zirconium, copper and nickel catalysts disclosed in U.S. Pat. No. 6,057,442 and suitable conditions include 80 to 300° C. and pressure of 0.1 to 40 MPa. Suitable catalysts and reaction conditions for this pathway are also disclosed in WO97/35834 and include contacting HDO and ammonia in the presence of a catalyst, that may include platinum, rhenium, cobalt, molybdenum, nickel, tungsten or palladium.

Once the conversions to HMD and ACN have been completed, it is also possible to convert those compounds into polyamide-type compounds in any number of ways known in the art. Representative examples include the following conversions. Polyamides may be produced from amino nitriles such as ACN. One example of conversions of this type may be found in U.S. Pat. No. 5,109,104 which converts an omega amino nitrile in the presence of an oxygenated phosphorus catalyst with water. This is generally achieved in a several-step conversion at temperatures of 200° C. to 330° C. and at pressures ranging from 1.72 to 2.41 MPa.

An alternative methodology is disclosed in U.S. Pat. No. 6,958,381 wherein a starting monomer such as ACN may be polymerized into the polyamide in the presence of a chain regulator containing a nitrile group and a functional group capable of forming a carboxamide group.

Polyamides may also be formed from the diamines such as HMD wherein the HMD is polymerized with a dicarboxylic acid or ester to form the polyamide. The preferred dicarboxylic acids have a chain length of C₄ to C₁₂. The dicarboxylic acid or ester may be an aromatic dicarboxylic acid or ester or it may be an alkyl dicarboxylic acid.

The subject matter and contents of the above-mentioned U.S. Pat. Nos. 4,237,067; 5,587,498; 3,296,303; 6,376,714; 4,003,933; 5,151,543; 7,132,562; 2,223,303; 3,579,583; 4,935,546; 5,109,104; and 6,958,381 are incorporated herein by reference.

### EXAMPLES

The processes are illustrated by the following non-limiting representative examples. In all examples, a synthetic, aqueous DAA solution was used in place of an actual clarified DAA-containing fermentation broth.

The use of a synthetic DAA solution is believed to be a good model for the behavior of an actual broth in our processes because of the solubility of the typical fermentation by-products found in actual broth. Typically, the major by-products produced during fermentation are salts of monocarboxylic acids such ammonium acetate, ammonium lactate and ammonium formate. If these impurities are present during the distillation step, one would not expect them to lose ammonia and form free acids in significant quantities until all of the DAA had been converted to MAA. This is because acetic acid, lactic acid and formic acid are stronger acids than the second acid group of AA (pKa=5.1). In other words, acetate, lactate, formate and even monohydrogen adipate are weaker bases than the dianion adipate. Furthermore, ammonium acetate, ammonium lactate and ammonium formate are significantly more soluble in water than MAA, and each is typically present in the broth at less than 10% of the DAA concentration. In addition, even if the acids (acetic, formic and lactic acids) were formed during the distillation step, they are miscible with water and will not crystallize from water. This means that the MAA reaches saturation and crystallizes from solution (i.e., forming the solid portion), leaving the acid impurities dissolved in the mother liquor (i.e., the liquid portion).

### Example 1

This example demonstrates conversion of a portion of DAA into MAA via distillation and recovery of MAA solids from distillation bottoms liquid via cooling-induced crystallization.

A 1-L round bottom flask was charged with 800 g of a synthetic 4.5% diammonium adipate (DAA) solution. The flask was fitted with a five tray 1″ Oldershaw section which was capped with a distillation head. The distillate was collected in an ice cooled receiver. The contents of the flask were heated with a heating mantel and stirred with a magnetic stirrer. Distillation was started and 719.7 g of distillate collected. Titration of the distillate revealed it was a 0.29% ammonia solution (i.e., an approximately 61% conversion of DAA to MAA). The hot residue (76 g) was discharged from the flask and placed in an Erlenmeyer flask and slowly cooled to room temperature while stirring over the weekend. The contents were then cooled to 15° C. for 60 minutes and then cooled to 10° C. for 60 minutes and finally 5° C. for 60 minutes while stirring. The solids were filtered and dried in a vacuum oven for 2 hours at 75° C. yielding 16.2 g. Analysis of the solids for ammonia content with an ammonia electrode indicated there was approximately a 1:1 molar ratio of ammonia and AA.

### Example 2

This example demonstrates conversion of a portion of DAA into MAA via distillation.

The outer necks of a three neck 1-L round bottom flask were fitted with a thermometer and a stopper. The center neck was fitted with a five tray I″ Oldershaw section. The Oldershaw section was topped with a distillation head. An ice cooled 500 mL round bottom flask was used as the receiver for the distillation head. The 1-L round bottom flask was charged with distilled water, AA and concentrated ammonium hydroxide solution. The contents were stirred with a magnetic stirrer to dissolve all the solids. After the solids dissolved, the contents were heated with the heating mantle to distill 350 g of distillate. The distillate was collected in the ice cooled 500 mL round bottom flask. The pot temperature was recorded as the last drop of distillate was collected. The pot contents were allowed to cool to room temperature and the weight of the residue and weight of the distillate were recorded. The ammonia content of the distillate was then determined via titration. The results were recorded in Table 1.

TABLE 1

| | |
|---|---|
| Run # | 1 |
| Name of Acid | Adipic |
| Wt Acid Charged (g) | 14.62 |
| Moles Acid Charged | 0.1 |
| Wt 28% NH₃ Solution Charged (g) | 12.14 |
| Moles NH₃ Charged | 0.2 |
| Wt Water Charged (g) | 800.75 |
| Wt Distillate (g) | 350.46 |
| Wt Residue (g) | 466.65 |
| % Mass Accountability | 98.8 |
| Wt % NH₃ in distillate (titration) | 0.15 |
| Moles NH₃ in distillate | 0.031 |
| % Total NH₃ removed in Distillate | 15.5 |
| % First NH₃ removed in Distillate | 31 |
| DiNH₄/MonoNH₄ | 69/31 |
| Final Pot Temp (° C.) | 100 |
| Micromoles of NH₃/g distillate | 89 |
| Initial Wt % ammonium salt | 2.2 |
| pKa₁ | 4.43 |
| pKa₂ | 5.41 |
| pKa₃ | NA |

### Example 3

This example demonstrates conversion of a portion of DAA into MAA in the presence of an ammonia releasing solvent via distillation and recovery of MAA solids from distillation bottoms liquid via cooling-induced crystallization.

A beaker was charged with 36.8 g of distilled water and 19.7 g of concentrated ammonium hydroxide. Then 23.5 g of adipic acid was slowly added. The mixture was stirred forming a clear solution which was then placed in a 500 mL round bottom flask which contained a stir bar. Triglyme (80 g) was then added to the flask. The flask was then fitted with a 5 tray 1″ Oldershaw column section which was topped with a distillation head. The distillation head was fitted with an ice bath cooled receiver. The distillation flask was also fitted with an addition funnel which contained 150 g of distilled water. The contents were then stirred and heated with a heating mantel. When distillate began to come over the water in the addition funnel was added dropwise to the flask at the same rate as the distillate take-off. The distillation was stopped when all of the water in the addition funnel had been added. A total of 158 g of distillate had been collected. Titration of the distillate revealed a 1.6% ammonia content. This is equivalent to 46% of the charged ammonia. In other words the residue is a 91/9 mixture of monoammonium adipate/diammonium adipate. After cooling to room temperature, the residue was place in a 250 mL Erlenmeyer flask and slowly cooled to 5° C. while stirring. The slurry was filtered and the wet crystals were then dried in a vacuum oven for 2 hours yielding 5.5 g of solids. Analysis of the solids indicated essentially a one to one ratio of ammonium ion to adipate ion (i.e. monoammonium adipate).

### Example 4

This example demonstrates the production of AA from MAA.

A 300 mL Parr autoclave was charged with 80 g of synthetic monoammonium adipate and 124 g of water. The autoclave was sealed and the contents stirred and heated to about 200° C. (autogenic pressure was about 203 psig). Once the contents reached temperature, water was then fed to the autoclave at a rate of about 2 g/min and vapor was removed from the autoclave at a rate of about 2 g/min with a back pressure regulator. Vapor exiting the autoclave was condensed and collected in a receiver. The autoclave was run under those conditions until a total of 1210 g of water had been fed and a total of 1185 g of distillate collected. The contents of the autoclave (209 g) were partially cooled and discharged from the reactor. The slurry was allowed to stand with stirring at room temperature over night in an Erlenmeyer flask. The slurry was then filtered and the solids rinsed with 25 g of water. The moist solids were dried in a vacuum oven at 75° C. for 1 hr yielding 59 g of adipic acid product. Analysis via an ammonium ion electrode revealed 0.015 mmole ammonium ion/g of solid. The melting point of the recovered solid was 151 to 154° C.

### Example 5

This example demonstrates conversion of a portion of MAA into AA in the presence of an ammonia releasing solvent via distillation and recovery of AA solids from distillation bottoms liquid via cooling-induced crystallization.

A beaker was charged with 46.7 g of distilled water and 9.9 g of concentrated ammonium hydroxide. Then 23.5 g of adipic acid was slowly added. The mixture was stirred forming a clear solution which was then placed in a 500 mL round bottom flask which contained a stir bar. Triglyme (80 g) was then added to the flask. The flask was then fitted with a 5 tray 1″ Oldershaw column section which was topped with a distillation head. The distillation head was fitted with an ice bath cooled receiver. The distillation flask was also fitted with an addition funnel which contained 1800 g of distilled water. The contents were then stirred and heated with a heating mantel. When distillate began to come over the water in the addition funnel was added dropwise to the flask at the same rate as the distillate take-off. The distillation was stopped when all of the water in the addition funnel had been added. A total of 1806.2 g of distillate had been collected. Titration of the distillate revealed a 0.11% ammonia content. This is equivalent to 72% of the charged ammonia. In other words the residue is a 72/28 mixture of adipic acid/monoammonium adipate. The residue was then placed in an Erlenmeyer flask and cooled to 0° C. while stirring and allowed to stand for 1 hr. The slurry was filtered yielding 18.8 g of a wet cake and 114.3 g of mother liquor. The solids were then dried under vacuum at 80° C. for 2 hrs yielding 13.5 g of solids. The solids were then dissolved in 114 g of hot water and then cooled to 5° C. and held stirring for 45 minutes.

The slurry was filtered yielding 13.5 g of wet solids and 109.2 g of mother liquor. The solids were dried under vacuum at 80° C. for 2 hrs yielding 11.7 g of dried solids. Analysis of the solids revealed an ammonium ion content of 0.0117 mmol/g (i.e. essentially pure adipic acid).Although our processes have been described in connection with specific steps and forms thereof, it will be appreciated that a wide variety of equivalents may be substituted for the specified elements and steps described herein without departing from the spirit and scope of this disclosure as described in the appended claims.

1. A process for producing nitrogen-containing compounds comprising:
(a) providing a clarified DAA-containing fermentation broth;
(b) distilling the broth to form an overhead that comprises water and ammonia, and a liquid bottoms that comprises MAA, at least some DAA, and at least about 20 wt % water;
(c) cooling and/or evaporating the bottoms, and optionally adding an antisolvent to the bottoms, to attain a temperature and composition sufficient to cause the bottoms to separate into a DAA-containing liquid portion and a MAA-containing solid portion that is substantially free of DAA;
(d) separating the solid portion from the liquid portion;
(e) recovering the solid portion;
(f) contacting at least a part of the solid portion at least one hydrogenation catalyst to produce HDO;
(g) aminating HDO to produce HMD; and
(h) recovering the HMD.

2. A process for making nitrogen-containing compounds comprising:
(a) providing a DAA-containing fermentation broth;
(b) distilling the broth to form a first overhead that comprises water and ammonia, and a first liquid bottoms that comprises MAA, at least some DAA, and at least about 20 wt % water;
(c) cooling and/or evaporating the bottoms, and optionally adding an antisolvent to the bottoms, to attain a temperature and composition sufficient to cause the bottoms to separate into a DAA-containing liquid portion and a MAA-containing solid portion that is substantially free of DAA;
(d) separating the solid portion from the liquid portion;
(e) recovering the solid portion;
(f) dissolving the solid portion in water to produce an aqueous MAA solution;
(g) distilling the aqueous MAA solution at a temperature and pressure sufficient to form a second overhead that comprises water and ammonia, and a second bottoms that comprises a major portion of AA, a minor portion of MAA, and water;
(h) cooling and/or evaporating the second bottoms to cause the second bottoms to separate into a second liquid portion in contact with a second solid portion that preferably consists essentially of AA and is substantially free of MAA;
(i) separating the second solid portion from the second liquid portion;
(j) recovering the second solid portion;
(k) contacting at least a part of the second solid portion with at least one hydrogenation catalyst to produce HDO;
(l) aminating the HDO to produce HMD; and
(m) recovering the HMD.

3. A process for making nitrogen-containing compounds comprising:
(a) providing a clarified MAA-containing fermentation broth;
(b) optionally, adding MAA, DAA, AA, NH₃, and/or NH₄⁺ to the broth to preferably maintain the pH of the broth below 6;
(c) distilling the broth to form an overhead that comprises water and optionally ammonia, and a liquid bottoms that comprises MAA, at least some DAA, and at least about 20 wt % water;
(d) cooling and/or evaporating the bottoms, and optionally adding an antisolvent to the bottoms, to attain a temperature and composition sufficient to cause the bottoms to separate into a DAA-containing liquid portion and a MAA-containing solid portion that is substantially free of DAA;
(e) separating the solid portion from the liquid portion;
(f) recovering the solid portion;
(g) contacting at least a part of the solid portion with at least one hydrogenation catalyst to produce HDO;
(h) aminating the HDO to produce HMD; and
(i) recovering the HMD.

4. A process for making nitrogen-containing compounds comprising:
(a) providing a clarified MAA-containing fermentation broth;
(b) optionally, adding MAA, DAA, AA, NH₃, and/or NH₄⁺ to the broth to preferably maintain the pH of the broth below 6;
(c) distilling the broth to form an overhead that comprises water and optionally ammonia, and a liquid bottoms that comprises MAA, at least some DAA, and at least about 20 wt % water;
(d) cooling and/or evaporating the bottoms, and optionally adding an antisolvent to the bottoms, to attain a temperature and composition sufficient to cause the bottoms to separate into a DAA-containing liquid portion and a MAA-containing solid portion that is substantially free of DAA;
(e) separating the solid portion from the liquid portion; and
(f) recovering the solid portion;
(g) dissolving the solid portion in water to produce an aqueous MAA solution;
(h) distilling the aqueous MAA solution at a temperature and pressure sufficient to form a second overhead that comprises water and ammonia, and a second bottoms that comprises a major portion of AA, a minor portion of MAA, and water;
(i) cooling and/or evaporating the second bottoms to cause the second bottoms to separate into a second liquid portion in contact with a second solid portion that preferably consists essentially of AA and is substantially free of MAA;
(j) separating the second solid portion from the second liquid portion;
(k) recovering the second solid portion;
(l) contacting at least a part of the second solid portion with one hydrogenation catalyst to produce HDO;
(m) aminating the HDO to produce HMD; and
(n) recovering the HMD.

5. The process of claim 1, wherein distilling the broth is carried out in the presence of an ammonia separating solvent which is at least one selected from the group consisting of diglyme, triglyme, tetraglyme, sulfoxides, amides, sulfones, polyethyleneglycol (PEG), butoxytriglycol, N-methylpyrolidone (NMP), ethers, and methyl ethyl ketone (MEK) or in the presence of a water azeotroping solvent which is at least one selected from the group consisting of toluene, xylene, methylcyclohexane, methyl isobutyl ketone, hexane, cyclohexane and heptane.

6. The process of claim 1, wherein dehydrating MAA is conducted thermally, catalytically or enzymatically.

7. The process of claim 2, wherein dehydrating AA is conducted thermally, catalytically or enzymatically.

8. The processes of claim 1 further comprising polymerizing the HMD with a dicarboxylic acid or ester to form a polyamide.

9. The process of claim 2, wherein distilling the broth is carried out in the presence of an ammonia separating solvent which is at least one selected from the group consisting of diglyme, triglyme, tetraglyme, sulfoxides, amides, sulfones, polyethyleneglycol (PEG), butoxytriglycol, N-methylpyrolidone (NMP), ethers, and methyl ethyl ketone (MEK) or in the presence of a water azeotroping solvent which is at least one selected from the group consisting of toluene, xylene, methylcyclohexane, methyl isobutyl ketone, hexane, cyclohexane and heptane.

10. The process of claim 3, wherein distilling the broth is carried out in the presence of an ammonia separating solvent which is at least one selected from the group consisting of diglyme, triglyme, tetraglyme, sulfoxides, amides, sulfones, polyethyleneglycol (PEG), butoxytriglycol, N-methylpyrolidone (NMP), ethers, and methyl ethyl ketone (MEK) or in the presence of a water azeotroping solvent which is at least one selected from the group consisting of toluene, xylene, methylcyclohexane, methyl isobutyl ketone, hexane, cyclohexane and heptane.

11. The process of claim 4, wherein distilling the broth is carried out in the presence of an ammonia separating solvent which is at least one selected from the group consisting of diglyme, triglyme, tetraglyme, sulfoxides, amides, sulfones, polyethyleneglycol (PEG), butoxytriglycol, N-methylpyrolidone (NMP), ethers, and methyl ethyl ketone (MEK) or in the presence of a water azeotroping solvent which is at least one selected from the group consisting of toluene, xylene, methylcyclohexane, methyl isobutyl ketone, hexane, cyclohexane and heptane.

12. The process of claim 3, wherein dehydrating MAA is conducted thermally, catalytically or enzymatically.

13. The process of claim 4, wherein dehydrating AA is conducted thermally, catalytically or enzymatically.

14. The processes of claim 2 further comprising polymerizing the HMD with a dicarboxylic acid or ester to form a polyamide.

15. The processes of claim 3 further comprising polymerizing the HMD with a dicarboxylic acid or ester to form a polyamide.

16. The processes of claim 4 further comprising polymerizing the HMD with a dicarboxylic acid or ester to form a polyamide.
